(19) United States
(12) Patent Application Publication
(10) Pub. No.: US 2022/0167863 A1
(43) Pub. Date: Jun. 2, 2022

(54) BLOOD VOLUME PULSE SIGNAL DETECTION APPARATUS, BLOOD VOLUME PULSE SIGNAL DETECTION APPARATUS METHOD, AND COMPUTER-READABLE STORAGE MEDIUM

(71) Applicant: NEC Corporation, Minato-ku, Tokyo (JP)

(72) Inventors: Jingwen LU, Tokyo (JP); Masanori TSUJIKAWA, Tokyo (JP)

(73) Assignee: NEC Corporation, Minato-ku, Tokyo (JP)

(21) Appl. No.: 17/441,364

(22) PCT Filed: Mar. 27, 2019
(86) PCT No.: PCT/JP2019/013425

§ 371 Date: Sep. 21, 2021

(51) Int. Cl. A61B 5/024; A61B 5/00

(52) U.S. Cl. CPC A61B 5/02416; A61B 5/7257; A61B 5/7485; A61B 5/7203

(57) ABSTRACT

A blood volume pulse signal detection apparatus 300 includes a ROI decision unit 325 that determines a ROI in input movie data including image of human face, a sub-ROI decision unit 340 that determines a sub-ROI based on the ROI, a filter design unit 330 that designs a bandpass filter by performing the analysis at frequency domain and/or time domain using an average blood volume pulse signals at ROI according to the physiological characteristics of heart rate, and a noise reduction unit 350 that enhances a blood volume pulse signal at each sub-ROI using the bandpass filter.

### TECHNICAL FIELD

The present invention relates to an apparatus and a method for robust physiological blood volume pulse detect from videos of human face, and a computer-readable storage medium storing a program for realizing these.

### BACKGROUND ART

Blood volume pulse signals occur when the blood volume in vessels changes with the heart beats. The blood volume pulse signals indicate relative changes in the vascular bed due to vasodilation or vasoconstriction as well as to changes in the elasticity of the vascular walls, which may be correlated with change in blood pressure. The blood pulse has been used to estimate the heart rate by the interval for the peaks in blood volume pulse signals. Hereinafter, “blood volume pulse” is denoted as “BVP”.

The peak-to-peak interval and amplitude are two important factors to understand BVP signals. The peak-to-peak interval of BVP signal has been used to evaluate the heart rate. In comparison, the BVP signal amplitude depends on placement of the sensor. It means if the spatial distribution of BVP signals can be detected, many biometric parameters can be relatively calculated such the blood vessel ages and temperature.

Usually BVP signals are detected by a PPG sensor. Recently, the BVP can also be detected by a visible webcam in a relatively larger area and make it possible to approach the spatial distribution of BVP easily (e.g., see Non Patent Literature 1).

### CITATION LIST

### Non Patent Literature

[NPL 1] Poh, Ming-Zher, Daniel J. McDuff, and Rosalind W. Picard, “Non-contact, automated cardiac pulse measurements using video imaging and blind source separation”, OPTICS EXPRESS Vol.18, No.10,2010.

### SUMMARY OF INVENTION

### Technical Problem

The first problem is that, it is more difficult to extract clean BVP signals at each sub region of interest which composes the spatial distribution of BVP in a region of interest, compared to the average BVP signals from region of interest of facial area. Because noise signals are relatively strong to BVP signals at each sub region of interest. Hereinafter, “region of interest” is denoted as “ROI”. “sub region of interest” is denoted as “sub-ROI”.

One of the objects of the present invention is to provide a blood volume pulse signal detection that is capable of extracting clean blood volume pulse signals at each sub-ROI.

### Solution to Problem

In order to achieve the foregoing object, a blood volume pulse signal detection apparatus includes:

a region of interest decision means that determines a region of interest in input movie data including image of human face,

a sub-ROI decision means that determines a sub-ROI based on the region of interest determined by the region of interest decision means,

a filter design means that designs a bandpass filter by performing the analysis at frequency domain and/or time domain using an average blood volume pulse signals at region of interest according to the physiological characteristics of heart rate,

a noise reduction means that enhances a blood volume pulse signal at each sub-ROI using the bandpass filter.

In order to achieve the foregoing object, blood volume pulse signal detection method includes:

(a) a step of determining a region of interest in input movie data including image of human face,

(b) a step of determining a sub-ROI based on the region of interest determined by the region of interest decision means,

(c) a step of designing a bandpass filter by performing the analysis at frequency domain and/or time domain using an average blood volume pulse signals at region of interest according to the physiological characteristics of heart rate,

(d) a step of enhancing a blood volume pulse signal at each sub-ROI using the bandpass filter.

In order to achieve the foregoing object, a computer-readable recording medium according to still another aspect of the present invention has recorded therein a program, and the program includes an instruction to cause a computer to execute:

(a) a step of determining a region of interest in input movie data including image of human face,

(b) a step of determining a sub-ROI based on the region of interest determined by the region of interest decision means,

(c) a step of designing a bandpass filter by performing the analysis at frequency domain and/or time domain using an average blood volume pulse signals at region of interest according to the physiological characteristics of heart rate,

(d) a step of enhancing a blood volume pulse signal at each sub-ROI using the bandpass filter.

### Advantageous Effects of Invention

As described above, according to the present invention, it is possible to extract clean blood volume pulse signals at each sub-ROI.

### BRIEF DESCRIPTION OF DRAWINGS

FIG. 1 A block diagram schematically showing the configuration of the BVP signal detection apparatus according to the embodiment of the present invention

FIG. 2 A block diagram showing the specific configuration of the BVP signal detection apparatus according to the embodiment of the present invention

FIG. 3 An example of ROI and sub-ROI referred in the embodiment of the present invention

FIG. 4 An example of average BVP signal obtained in ROI at time and frequency domain referred in the embodiment of the present invention

FIG. 5 An example of BVP signal obtained in each sub-ROI at time and frequency domain referred in the embodiment of the present invention

FIG. 6 An example of BVP signal obtained in each sub-ROI at frequency domain after noise reduction in the embodiment of the present invention

FIG. 7 An example of BVP spatial distribution at certain time extracted by the present invention

FIG. 8 A flowchart showing operations performed by the BVP signal detection apparatus according to the embodiment of the present invention

FIG. 9 A block diagram showing an example of a computer that realizes the BVP signal detection apparatus according to an embodiment of the present invention

### DESCRIPTION OF EMBODIMENTS

### Embodiment

Hereinafter, an exemplary embodiment of the current invention will be described in detail. The implementation is described in complete detail referring to the accompanying drawings.

### Device Configuration

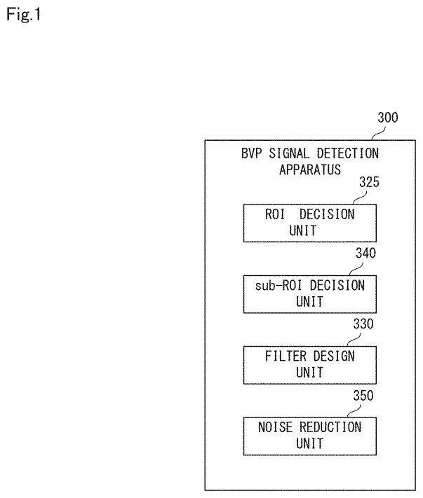

First, a configuration of a BVP signal detection apparatus according to the present embodiment will be described using FIG. 1. FIG. 1 is a block diagram schematically showing the configuration of the BVP signal detection apparatus according to the embodiment of the present invention.

A BVP signal detection apparatus 300 shown in FIG. 1 is an apparatus for detecting BVP signals. As shown in FIG. 1, the BVP signal detection apparatus 300 includes a ROI decision unit 325, a sub-ROI decision unit 340, a filter design unit 330, and a noise reduction unit 350.

The ROI decision unit 325 determines a ROI in input movie data including image of human face. The sub-ROI decision unit 340 determines a sub-ROI based on the ROI determined by the ROI decision unit 325.

The filter design unit 330 designs a bandpass filter by performing the analysis at frequency domain and/or time domain using average BVP signals at ROI according to the physiological characteristics of heart rate. The noise reduction unit 350 enhances a BVP signal at each sub-ROI using the bandpass filter.

As described above, in the present embodiment, the frequency domain and/or time domain analysis using the average BVP signal at ROI is performed to design a filter, and the BVP signal at each sub-ROI is enhanced by this filter. As a result, it is possible to extract clean BVP signals at each sub-ROI.

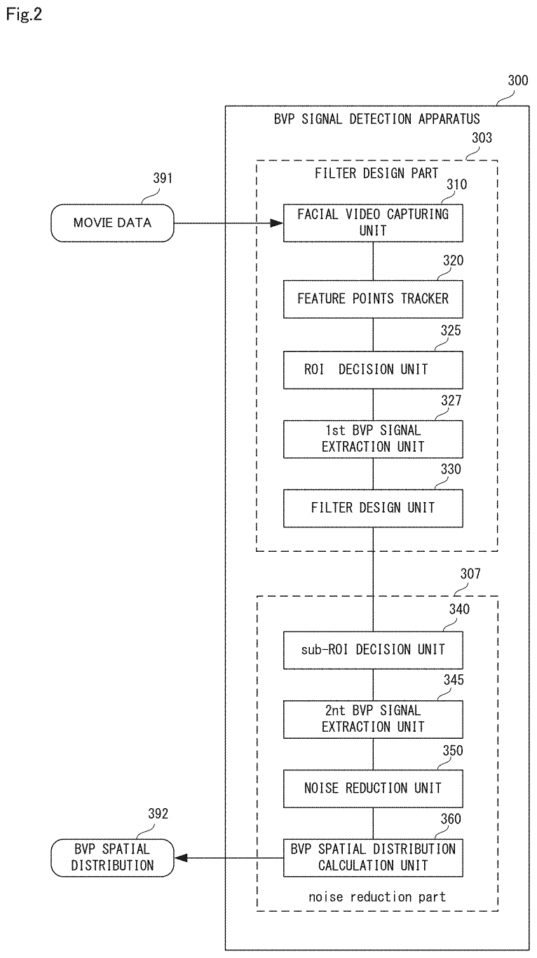

Next, the configuration and function of the BVP signal apparatus of the embodiment will be described in detail with reference to FIGS. 2 to 7 as well. FIG. 2 is a block diagram showing the specific configuration of the BVP signal detection apparatus according to the embodiment of the present invention.

As shown in FIG. 2, in this embodiment, the BVP signal detection apparatus 300 further includes a facial video capturing unit 310, a feature points tracker 320, a first BVP signal extraction unit 327, a second BVP signal extraction unit 345, and BVP spatial distribution calculation unit 360, in addition to the ROI decision unit 325, the sub-ROI decision unit 340, the filter design unit 330, and the noise reduction unit 350.

Further, as shown in FIG. 2, the BVP signal detection apparatus 300 in this embodiment can be broadly divided into 2 parts, which are the filter design part 303 and the noise reduction part 307 as shown in FIG. 2.

For the filter design part, it is composed by the facial video capturing unit 310, the feature points tracker 320, the ROI decision unit 325, the first BVP signal extraction unit 327, and the filter design unit 330. For the noise reduction part, it includes the sub-ROI decision unit 340, second BVP signal extraction unit 345, noise reduction unit 350, and the BVP spatial distribution calculation unit 360.

The facial video capturing unit 310 captures a human face image from the input movie data 391. The feature point tracker 320 track the face and output the feature points in each frame of the input movie data 391. Based on the facial feature points, the ROI decision unit 325 selects and decides the ROI, and at the same time sub-ROI is localized by the sub-ROI decision unit 340 as well.

After getting the feature points and deciding the ROI, the 1st BVP signal extraction unit 327 obtains the BVP signals by reading the RGB values at each frame. The first BVP signal extraction unit 327 calculates the average BVP signals in the ROI using the follows Math. 1.

$${BVP}_{ROI} = \frac{1}{m} {\sum }_{pi = 1}^{m} {BVP}_{pi} \qquad [ Math . 1 ]$$

In the Math. 1, BVP_{ROI} is the average BVP signal in ROI, BVPₚᵢ is the BVP signals obtained at pixel i, m is the total numbers of pixels in ROI, pi is the sequence number of pixels. According to Math. 1, the average BVP signal in ROI is obtained by spatially averaging BVP signal over all pixels in the ROI for each frame. This means that the average BVP in ROI is a combined signal, where the noise caused by artifacts, light and facial expression is statistically smoothed

The filter design unit 330 performs the frequency and/or time domain analysis by executing Fourier transform using BVP_{ROI} during certain period. According to the frequency and/or time domain analysis of BVP_{ROI}, the spectral peak in the power spectrum of BVP_{ROI} is tracked to select an appropriate BVP frequency range as a filter. The selected appropriate BVP frequency range will be used to enhance the BVP signal at each sub-ROI by the noise reduction unit 350.

Note that this filter is different from the operational frequency extracted by experience. The filter extracted from operational frequency lies on a relatively larger range, such as from the range of 0.25 Hz to 4 Hz according the range of heart rate of 15-240 beats per minute for general human, while the filter extracted from the filter design unit 330 is a dynamic filter, which allow the noise to be reasonably eliminated but does not distort BVP signal significantly compared to the filter from operational frequency. The filter from operational frequency could be used as an auxiliary parameter to estimate if the ROI-extracted filter is reasonable.

Based on the ROI of the captured facial, the sub-ROI decision unit 340 decides the sub-ROI by dividing the ROI into smaller sub-ROI. The sub-ROI demonstrates the resolution of the BVP spatial distribution. Note that it is no need to get sub-ROI by dividing ROI evenly. Sub-ROI can be any shape, each of which represents part of ROI, and all of which compose the ROI.

After the sub-ROI is determined by the sub-ROI decision unit 340, the second BVP signal extraction unit 345 extracts the BVP signal at each sub-ROI. The methods of extracting the BVP signal at each sub-ROI is similar to the methods of gaining the average BVP signal in the ROI. The BVP signal at each sub-ROI is spatial average of BVP signal over all of the pixels in the range of sub-ROI.

The noise reduction unit 350 enhances the detected BVP signal at each sub-ROI. One example lies on the noise reduction at frequency domain, where Fourier transform can be applied to the detected BVP signal with certain time at each sub-ROI. According to the filter drawn from the filter design unit 330, only BVP signals at certain frequency range can be passed after applying the filter.

Another example lies on the noise reduction at time domain, where the filter at time domain can be applied to BVP signal at each sub-ROI. This filter is also designed by the filter design unit 330. After applied the filter at frequency and/or time domain, the clean signal for each sub-ROI is obtained.

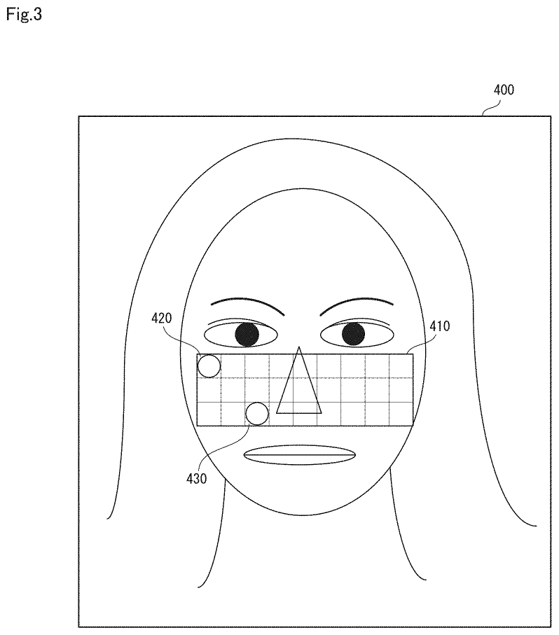

FIG. 3 shows an example of ROI and sub-ROI referred in the embodiment of the present invention. In FIG. 3, reference numeral 400 denotes a frame, and reference numeral 410 denotes an ROI. Examples 420 and 430 in FIG. 3 showed an example of sub-ROI. BVP signals detected from each sub-ROI are analyzed with respect of time domain and/or frequency domain by Fourier transform, and then the power spectrum in frequency domain are obtained.

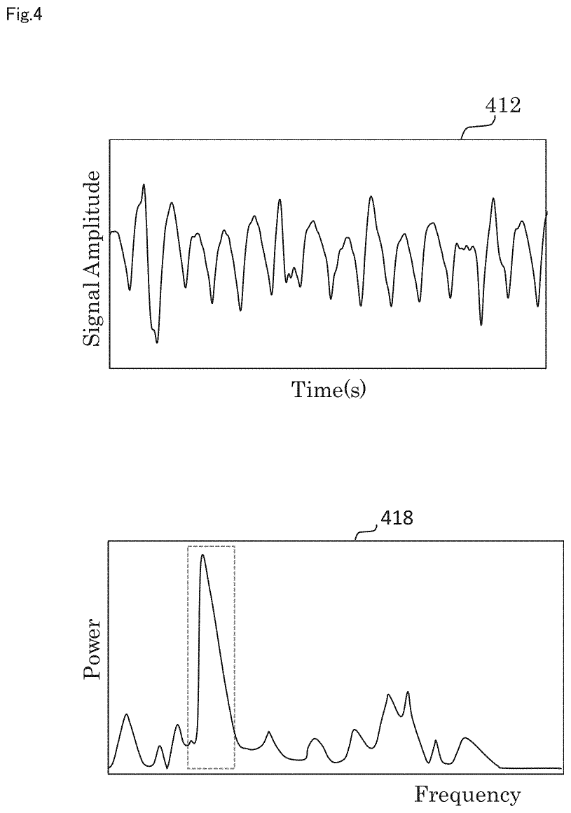

FIG. 4 shows an example of average BVP signal obtained in ROI at time and frequency domain referred in the embodiment of the present invention. For example, consider 412 as the average BVP signals obtained in ROI, and 418 is its power spectrum at frequency domain of the average BVP signals in ROI. According to the filter design unit 330, the BVP signals with time as shown in 412 are changed to the power spectrum in frequency domain, as shown in 418. It could be observed that there is a region designated by dash rectangle with narrow peak shown in 418, which is considered to correspond as the main BVP signals. Based on this power spectrum analysis, the bandpass filter will be extracted as a filter for noise reduction. Note that the bandpass should not conflict with the operational frequency, which lies on a relatively larger range, such as from the range of 0.25 Hz to 4 Hz according to the range of heart rate of 15-240 beats per minute for general human.

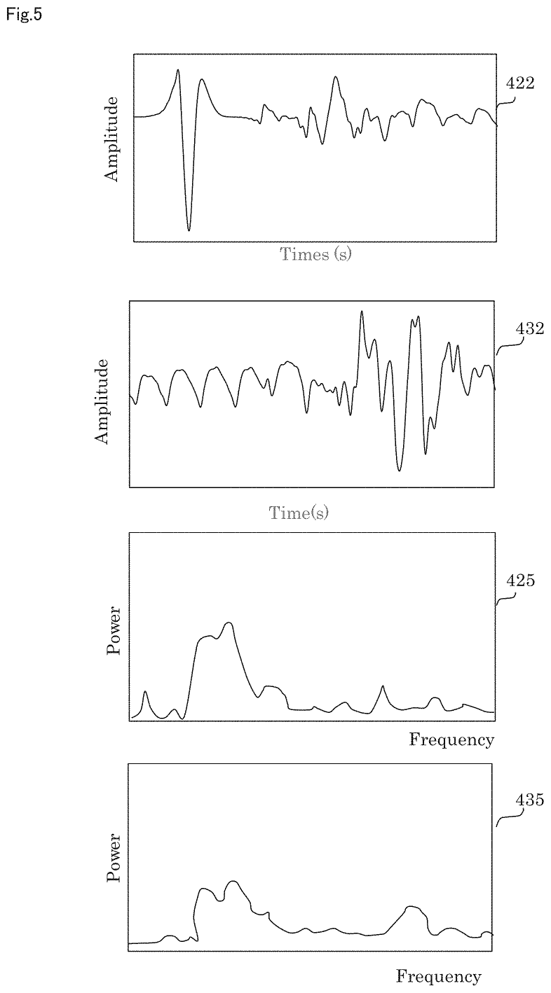

FIG. 5 shows an example of BVP signal obtained in each sub-ROI at time and frequency domain referred in the embodiment of the present invention. For example, consider 422 and 432 in FIG. 5 as a set of BVP signals at time domain obtained from sub-ROI 420 and sub-ROI 430, and 425 and 435 in FIG. 5 are the power spectrum in frequency domain correspondingly. The amplitude in 425 and 435 spreads in a larger range, and the peak of amplitude is not very clear compared to 418, which is the powerspectrum of the average BVP in ROI.

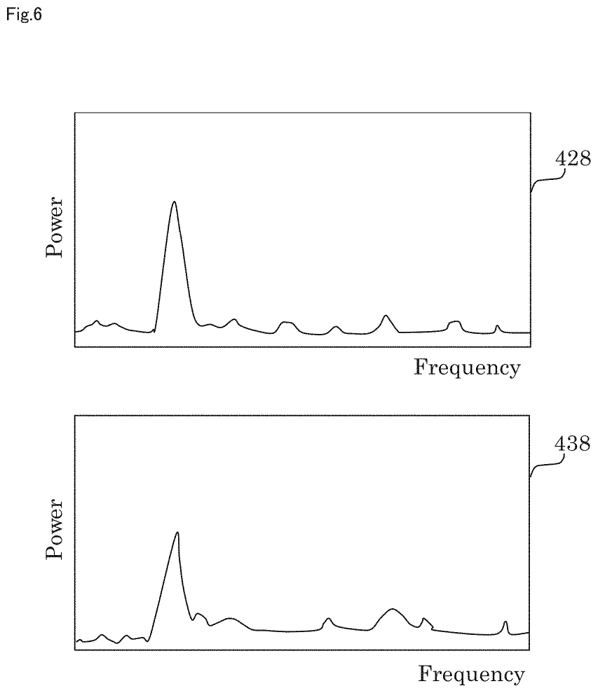

FIG. 6 shows an example of BVP signal obtained in each sub-ROI at frequency domain after noise reduction in the embodiment of the present invention. An example of the noise-reduced output spectrum for the noisy signal of 425 and 435 in is in shown in 428 and 438 in FIG. 6 Note that because some of the desired signal spectral components were below the noise threshold of the bandpass filer, the spectral subtraction process inadvertently removes them. Nevertheless, the spectral subtraction method can conceivably improve the signal-to-noise ratio.

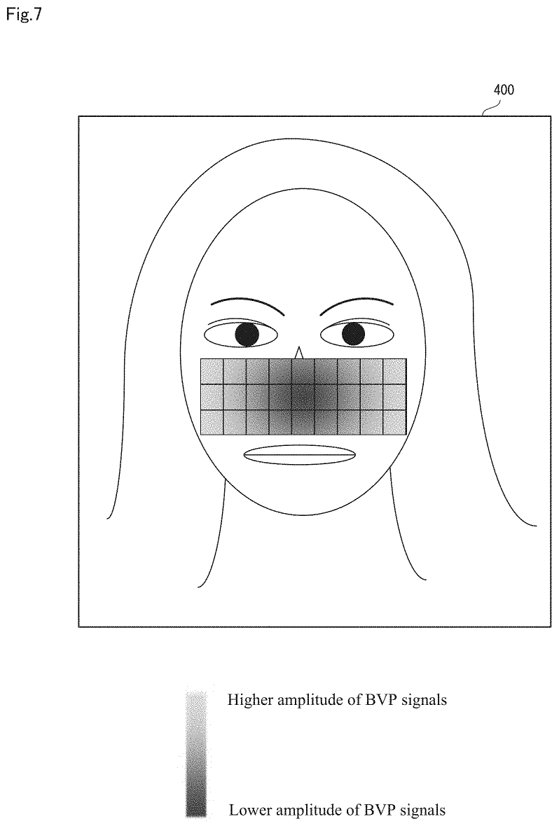

The BVP spatial distribution calculation unit 360 calculates a BVP spatial distribution from the filtering BVP signal at each sub-ROI. The BVP spatial distribution calculation unit 360 output the BVP spatial distribution 392 to outside device. FIG. 7 shows an example of BVP spatial distribution at certain time extracted by the present invention.

For example, the BVP spatial distribution at ROI at certain frame could be obtained by this embodiment. The BVP spatial distribution can be expressed as the shown in FIG. 7. According to the calculated BVP spatial distribution, other physiological information could be further calculated.

### Operations of Apparatus

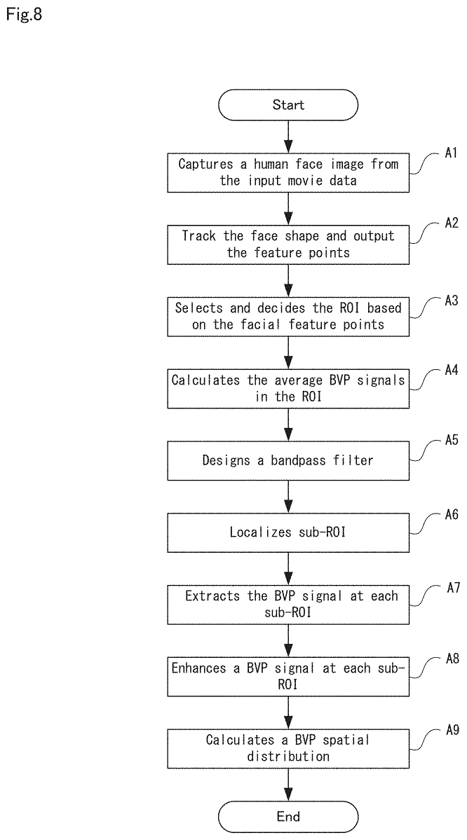

Next, operations performed by the BVP signal detection apparatus 300 according to the embodiment of the present invention will be described with reference to FIG. 8. FIG. 8 is a flowchart showing operations performed by the BVP signal detection apparatus according to the embodiment of the present invention. FIG. 1-7 will be referred to as needed in the following description.

Also, in the present embodiment, the BVP signal detection method is carried out by allowing the BVP signal detection apparatus 300 to operate. Accordingly, the description of the BVP signal detection method of this embodiment will be substituted with the following description of operations performed by the BVP signal detection apparatus 300.

First, as shown FIG. 8, the facial video capturing unit 310 captures a human face image from the input movie data 391 (step A1).

Next, the feature point tracker 320 track the face and output the feature points in each frame of the input movie data 391 (step A2).

Next, the ROI decision unit 325 selects and decides the ROI based on the facial feature points output in step A2 (step A3).

Next, the first BVP signal extraction unit 327 calculates the average BVP signals in the ROI selected and decided in step A3, using the above Math. 1 (step A4).

Next, the filter design unit 330 designs a bandpass filter by performing the analysis at frequency domain and/or time domain using average BVP signals calculated in step A4, at ROI (step A5).

Next, the sub-ROI decision unit 340 localizes sub-ROI as well as step A3 (step A6).

Next, the sub-ROI is determined by the sub-ROI decision unit 340 in step A6, the second BVP signal extraction unit 345 extracts the BVP signal at each sub-ROI (step A7).

Next, the noise reduction unit 350 enhances a BVP signal at each sub-ROI using the bandpass filter designed in the step A5 (step A8). As a result, the noise in the BVP signal is reduced.

Next, the BVP spatial distribution calculation unit 360 calculates a BVP spatial distribution from the BVP signal processed in step A8, at each sub-ROI (step A9). After that, the BVP spatial distribution calculation unit 360 output the BVP spatial distribution 392 to outside device.

### Effects of the Present Embodiment

A first effect is to ensure that it is possible to extract clean BVP signals at each sub-ROI. Because, the frequency domain and/or time domain analysis using the average BVP signal is performed to design a filter, and the BVP signal is enhanced by this filter.

A second effect is to ensure that it is possible to accurately detect a BVP spatial distribution at certain time because, clean BVP signals are extracted at each sub-ROI. As a result, it is possible to read a great deal of biometric information from the spatial distribution of BVP signals.

### Program

A program of the present embodiment need only be a program for causing a computer to execute steps A1 to A9 shown in FIG. 8. The BVP signal detection apparatus and the BVP signal detection method according to the present embodiment can be realized by installing the program on a computer and executing it. In this case, the Processor of the computer functions as the facial video capturing unit 310, the feature points tracker 320, the ROI decision unit 325, a first BVP signal extraction unit 327, the filter design unit 330, the sub-ROI decision unit 340, the second BVP signal extraction unit 345, the noise reduction unit 350, and the BVP spatial distribution calculation unit 360, and performs processing.

The program according to the present exemplary embodiment may be executed by a computer system constructed using a plurality of computers. In this case, for example, each computer may function as a different one of the facial video capturing unit 310, the feature points tracker 320, the ROI decision unit 325, a first BVP signal extraction unit 327, the filter design unit 330, the sub-ROI decision unit 340, the second BVP signal extraction unit 345, the noise reduction unit 350, and the BVP spatial distribution calculation unit 360.

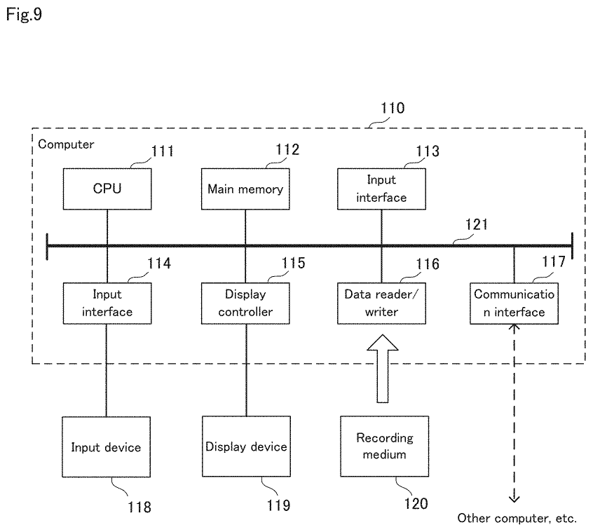

Also, a computer that realizes the BVP signal detection apparatus 300 by executing the program according to the present embodiment will be described with reference to the drawings. FIG. 9 is a block diagram showing an example of a computer that realizes the BVP signal detection apparatus according to an embodiment of the present invention.

As shown in FIG. 9, the computer 110 includes a CPU (Central Processing Unit) 111, a main memory 112, a storage device 113, an input interface 114, a display controller 115, a data reader/writer 116, and a communication interface 117. These units are connected via a bus 121 so as to be capable of mutual data communication. The computer 110 may include a GPU (Graphics Processing Unit) or FPGA (Field-Programmable Gate Array) in addition to or in place of the CPU 111.

The CPU 111 carries out various calculations by expanding programs (codes) according to the present embodiment, which are stored in the storage device 113, to the main memory 112 and executing them in a predetermined sequence. The main memory 112 is typically a volatile storage device such as a DRAM (Dynamic Random Access Memory). Also, the program according to the present embodiment is provided in a state of being stored in a computer-readable storage medium 120. Note that the program according to the present embodiment may be distributed over the Internet, which is connected to via the communication interface 117.

Also, specific examples of the storage device 113 include a semiconductor storage device such as a flash memory, in addition to a hard disk drive. The input interface 114 mediates data transmission between the CPU 111 and an input device 118 such as a keyboard or a mouse. The display controller 115 is connected to a display device 119 and controls display on the display device 119.

The data reader/writer 116 mediates data transmission between the CPU 111 and the storage medium 120, reads out programs from the storage medium 120, and writes results of processing performed by the computer 110 in the storage medium 120. The communication interface 117 mediates data transmission between the CPU 111 and another computer.

Also, specific examples of the storage medium 120 include a general-purpose semiconductor storage device such as CF (Compact Flash (registered trademark)) and SD (Secure Digital), a magnetic storage medium such as a flexible disk, and an optical storage medium such as a CD-ROM (Compact Disk Read Only Memory).

The BVP signal detection apparatus 300 according to the present exemplary embodiment can also be realized using items of hardware corresponding to various components, rather than using the computer having the program installed therein. Furthermore, a part of the BVP signal detection apparatus 300 may be realized by the program, and the remaining part of the BVP signal detection apparatus 300 may be realized by hardware.

The above-described embodiment can be partially or entirely expressed by, but is not limited to, the following Supplementary Notes 1 to 9.

### Supplementary Note 1

A blood volume pulse signal detection apparatus comprising:

a ROI decision means that determines a ROI in input movie data including image of human face,

a sub-ROI decision means that determines a sub-ROI based on the ROI determined by the ROI decision means,

a filter design means that designs a bandpass filter by performing the analysis at frequency domain and/or time domain using an average blood volume pulse signals at ROI according to the physiological characteristics of heart rate,

a noise reduction means that enhances a blood volume pulse signal at each sub-ROI using the bandpass filter.

### Supplementary Note 2

The blood volume pulse signal detection apparatus according to supplementary note 1, further comprising,

A blood volume pulse spatial distribution calculation means calculates blood volume pulse spatial distribution from the filtered blood volume pulse signal at each sub-ROI.

### Supplementary Note 3

The blood volume pulse signal detection apparatus according to supplementary note 2,

wherein the blood volume pulse spatial distribution calculation means decides the resolution of blood volume pulse spatial distribution using the sub-ROI.

### Supplementary Note 4

The blood volume pulse signal detection apparatus according to any of supplementary notes 1 to 3,

Wherein the filter design means designs the bandpass filter to enhance the blood volume pulse signals at each sub-ROI, whose upper and lower cut frequencies are determined by analysis of blood volume pulse signal with respect of time and/or frequency domain obtained in the ROI and the physiological characteristics of human's general heart rate.

### Supplementary Note 5

The blood volume pulse signal detection apparatus according to supplementary note 2 or 3,

wherein the blood volume pulse spatial distribution calculation means calculates the blood volume pulse spatial distribution by extracting blood volume pulse signals at each sub-ROI, which have been enhanced by using the bandpass filters to reduce noise.

### Supplementary Note 6

A blood volume pulse signal detection method comprising:

(a) a step of determining a ROI in input movie data including image of human face,

(b) a step of determining a sub-ROI based on the ROI determined by the ROI decision means,

(c) a step of designing a bandpass filter by performing the analysis at frequency domain and/or time domain using an average blood volume pulse signals at ROI according to the physiological characteristics of heart rate,

(d) a step of enhancing a blood volume pulse signal at each sub-ROI using the bandpass filter.

### Supplementary Note 7

The blood volume pulse signal detection method according to supplementary note 6, further comprising,

(e) a step of calculating a blood volume pulse spatial distribution from the filtered blood volume pulse signal at each sub-ROI.

### Supplementary Note 8

The blood volume pulse signal detection method according to supplementary note 7, further comprising,

(f) a step of deciding the resolution of blood volume pulse spatial distribution using the sub-ROI.

### Supplementary Note 9

The blood volume pulse signal detection method according to any of supplementary notes 6 to 8,

Wherein in the step (c), designing the bandpass filter to enhance the blood volume pulse signals at each sub-ROI, whose upper and lower cut frequencies are determined by analysis of blood volume pulse signal with respect of time and/or frequency domain obtained in the ROI and the physiological characteristics of human's general heart rate.

### Supplementary Note 10

The blood volume pulse signal detection method according to supplementary note 7 or 8,

wherein in the step (e), calculating the blood volume pulse spatial distribution by extracting blood volume pulse signals at each the sub-ROI, which have been enhanced by using the bandpass filters to reduce noise.

### Supplementary Note 11

A computer-readable medium having recorded thereon a program, the program including instructions for causing a computer to execute:

(a) a step of determining a ROI in input movie data including image of human face,

(b) a step of determining a sub-ROI based on the ROI determined by the ROI decision means,

(c) a step of designing a bandpass filter by performing the analysis at frequency domain and/or time domain using an average blood volume pulse signals at ROI according to the physiological characteristics of heart rate,

(d) a step of enhancing a blood volume pulse signal at each sub-ROI using the bandpass filter.

### Supplementary Note 12

The computer-readable medium according to supplementary note 11, the program further including instruction for causing a computer to execute:

(e) a step of calculating a blood volume pulse spatial distribution from the filtered blood volume pulse signal at each sub-ROI.

### Supplementary Note 13

The computer-readable medium according to supplementary note 12, the program further including instruction for causing a computer to execute:

(f) a step of deciding the resolution of blood volume pulse spatial distribution using the sub-ROI.

### Supplementary Note 14

The computer-readable medium according to any of supplementary notes 11 to 13,

Wherein in the step (c), designing the bandpass filter to enhance the blood volume pulse signals at each sub-ROI, whose upper and lower cut frequencies are determined by analysis of blood volume pulse signal with respect of time and/or frequency domain obtained in the ROI and the physiological characteristics of human's general heart rate.

### Supplementary Note 15

The computer-readable medium according to supplementary note 12 or 13,

wherein in the step (e), calculating the blood volume pulse spatial distribution by extracting blood volume pulse signals at each sub-ROI, which have been enhanced by using the bandpass filters to reduce noise.

Although the invention of the present application has been described above with reference to the embodiment, the invention of the present application is not limited to the above embodiment. Various changes that can be understood by a person skilled in the art can be made to the configurations and details of the invention of the present application within the scope of the invention of the present application.

### INDUSTRIAL APPLICABILITY

As described above, according to the present invention, it is possible to extract clean BVP signals at each sub-ROI. The present invention is useful in fields detecting robust physiological blood volume pulse signals.

### REFERENCE SIGNS LIST

110 Computer
111 CPU
112 Main memory
113 Storage device
114 Input interface
115 Display controller
116 Data reader/writer
117 Communication interface
118 Input device
119 Display apparatus
120 Storage medium
121 Bus
300 BVP signal detection apparatus
310 facial video capturing unit
320 feature points tracker 320
325 ROI decision unit
327 first BVP signal extraction unit
330 filter design unit
340 sub-ROI decision unit
345 second BVP signal extraction unit
350 noise reduction unit
360 BVP spatial distribution calculation unit

What is claimed is:

1. A blood volume pulse signal detection apparatus comprising:
a ROI decision unit configured to determine a ROI in input movie data including image of human face,
a sub-ROI decision unit configured to determine a sub-ROI based on the ROI determined by the ROI decision means,
a filter design unit configured to design a bandpass filter by performing the analysis at frequency domain and/or time domain using an average blood volume pulse signals at ROI according to the physiological characteristics of heart rate,
a noise reduction unit configured to enhance a blood volume pulse signal at each sub-ROI using the bandpass filter.

2. The blood volume pulse signal detection apparatus according to claim 1, further comprising,
a blood volume pulse spatial distribution calculation unit calculates a blood volume pulse spatial distribution from the enhanced blood volume pulse signal at each sub-ROI.

3. The blood volume pulse signal detection apparatus according to claim 2,
wherein the blood volume pulse spatial distribution calculation unit determines the resolution of blood volume pulse spatial distribution using the sub-ROI.

4. The blood volume pulse signal detection apparatus according to claim 1,
wherein the filter design unit designs the bandpass filter to enhance the blood volume pulse signals at each sub-ROI, whose upper and lower cut frequencies are determined by analysis of blood volume pulse signal with respect of time and/or frequency domain obtained in the ROI and the physiological characteristics of human's general heart rate.

5. The blood volume pulse signal detection apparatus according to claim 2,
wherein the blood volume pulse spatial distribution calculation unit calculates the blood volume pulse spatial distribution by extracting blood volume pulse signals at each sub-ROI, which have been enhanced by using the bandpass filters to reduce noise.

6. A blood volume pulse signal detection method comprising:
determining a ROI in input movie data including image of human face,
determining a sub-ROI based on the ROI determined by determining the ROI,
designing a bandpass filter by performing the analysis at frequency domain and/or time domain using an average blood volume pulse signals at ROI according to the physiological characteristics of heart rate,
enhancing a blood volume pulse signal at each sub-ROI using the bandpass filter.

7. A non-transitory computer-readable medium having recorded thereon a program, the program including instructions for causing a computer to execute:
determining a ROI in input movie data including image of human face,
determining a sub-ROI based on the ROI determined by determining the ROI,
designing a bandpass filter by performing the analysis at frequency domain and/or time domain using an average blood volume pulse signals at ROI according to the physiological characteristics of heart rate,
enhancing a blood volume pulse signal at each sub-ROI using the bandpass filter.

8. The blood volume pulse signal detection method according to claim 6, further comprising,
calculating a blood volume pulse spatial distribution from the enhanced blood volume pulse signal at each sub-ROI.

9. The blood volume pulse signal detection method according to claim 8, further comprising,
determining the resolution of blood volume pulse spatial distribution using the sub-ROI.

10. The blood volume pulse signal detection method according to claim 6,
wherein designing the bandpass filter to enhance the blood volume pulse signals at each sub-ROI, whose upper and lower cut frequencies are determined by analysis of blood volume pulse signal with respect of time and/or frequency domain obtained in the ROI and the physiological characteristics of human's general heart rate.

11. The blood volume pulse signal detection method according to claim 8,
wherein calculating the blood volume pulse spatial distribution by extracting blood volume pulse signals at each the sub-ROI, which have been enhanced by using the bandpass filters to reduce noise.

12. The non-transitory computer-readable medium according to claim 7, the program further including instruction for causing a computer to execute:
calculating a blood volume pulse spatial distribution from the enhanced blood volume pulse signal at each sub-ROI.

13. The non-transitory computer-readable medium according to claim 12, the program further including instruction for causing a computer to execute:
determining the resolution of blood volume pulse spatial distribution using the sub-ROI.

14. The non-transitory computer-readable medium according to claim 7,
wherein designing the bandpass filter to enhance the blood volume pulse signals at each sub-ROI, whose upper and lower cut frequencies are determined by analysis of blood volume pulse signal with respect of time and/or frequency domain obtained in the ROI and the physiological characteristics of human's general heart rate.

15. The non-transitory computer-readable medium according to claim 12,
wherein calculating the blood volume pulse spatial distribution by extracting blood volume pulse signals at each sub-ROI, which have been enhanced by using the bandpass filters to reduce noise.
